(19) United States
(12) Patent Application Publication
(10) Pub. No.: US 2026/0041415 A1
(43) Pub. Date: Feb. 12, 2026

(54) TISSUE TRACTION DEVICES, SYSTEMS, AND METHODS

(71) Applicant: BOSTON SCIENTIFIC SCIMED, INC., MAPLE GROVE, MN (US)

(72) Inventors: Gonzalo Jose Saenz Villalobos, Alajuela (CR); Yeison Calvo, San Ramón (CR); Gian Franco Loo Fuchs, San Jose (CR)

(73) Assignee: BOSTON SCIENTIFIC SCIMED, INC., MAPLE GROVE, MN (US)

(21) Appl. No.: 19/102,274

(22) PCT Filed: Aug. 8, 2023
(86) PCT No.: PCT/US2023/029687

§ 371 Date: Feb. 7, 2025

Related U.S. Application Data
(60) Provisional application No. 63/396,380, filed on Aug. 9, 2022.

(51) Int. Cl. A61B 17/02; A61B 1/018; A61B 17/00

(52) U.S. Cl. CPC A61B 17/0218; A61B 1/018; A61B 17/0206; A61B 2017/00477

(57) ABSTRACT

A tissue traction device with a pair of legs biased with respect to each other by a biasing section. The legs may be biased apart and engaged with tissue sites to be separated from each other upon the legs being biased apart from each other. Alternatively, the legs may be biased together, and one leg engaged with tissue cut with respect to a treatment site and the other leg engaged with anchoring tissue spaced apart from the treatment site. The legs are biased together to draw the tissue cut with respect to the treatment site away from the treatment site and towards the anchoring tissue. The tissue traction device may be included in a tissue traction system having a delivery shaft configured to deliver the tissue traction device to a treatment site.

### CROSS-REFERENCE TO RELATED APPLICATIONS

This application claims the benefit of priority to U.S. Provisional Application No. 63/396,830, filed Aug. 9, 2022, the entire disclosure of which is hereby incorporated by reference herein for all purposes.

### FIELD

The present disclosure relates generally to the field of devices, systems, and methods for applying traction to tissue. More particularly, the present disclosure relates to a traction device and system, and use of the device and system to extend tissue away from a treatment site.

### BACKGROUND

Various endoscopic surgical procedures require maneuvering about various anatomical structures. Some procedures, such as endoscopic mucosal resection (EMR), Endoscopic Submucosal Dissection (ESD), Pre-Oral Endoscopic Myotomy (POEM), etc., allow for minimally invasive endoscopic removal of benign and early malignant lesions, such as in the gastrointestinal (GI) tract. Minimally-invasive surgical techniques like these typically allow for faster recovery than with open or laparascopic surgical procedures. However, because such procedures are minimally invasive, there is limited space to maneuver within the body, and such procedures typically require a high degree of expertise. In procedures involving cutting of tissue, one of the largest time and complexity drivers is managing the tissue being cut. The loose section of tissue may obstruct visibility, such as by falling on the endoscope, occluding visibility of the camera and creating a hindrance affecting movement of the instruments used during the procedure and in reaching all regions and depths of the tissue being cut at the treatment site. Various solutions for lifting the cut (and often hanging) mass of tissue, thus clearing the path for visibility and operation of medical tools and devices, have been developed. However, positioning of the elements used with such solutions may be challenging, particularly in a space-restricted environment. Also, the elements used with such solutions may require separate medical tools than those used to perform the procedure, and such tools may even require a separate working channel in the endoscope, thereby potentially increasing the size and/or complexity of the endoscope. Alternative solutions for lifting tissue during a procedure which reduce cost, complexity, and cognitive load presented by currently available solutions would be welcome.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

In accordance with various principles of the present disclosure, a tissue traction system includes a pair of legs biased with respect to each other by a biasing section; and a delivery shaft defining a lumen within which the tissue traction device is deliverable to a treatment site. In some aspects, the tissue traction device is shiftable between a delivery configuration and a deployment configuration; the tissue traction device legs include tissue-engaging tips configured to engage tissue; the delivery shaft is configured to maintain the tissue traction device in the delivery configuration; and in the deployment configuration, the biasing section of the tissue traction device exerts traction on tissue through the tissue traction device legs to draw tissue away from a treatment site.

In some embodiments, the biasing section joins the legs of the tissue traction device together.

In some embodiments, the biasing section biases the legs of the tissue traction device away from each other to lift tissue away from a treatment site.

In some embodiments, the biasing section biases the legs of the tissue traction device towards each other to lift tissue away from a treatment site and towards anchoring tissue spaced apart from the treatment site.

In some embodiments, at least one of the tissue-engaging tips extends from one of the legs transverse to the one of the legs and in a direction in which the biasing section biases the one of the legs. In some embodiments, the at least one of the tissue-engaging tip extends from a free end of the one of the legs. In some embodiments, the biasing section biases the legs of the tissue traction device away from each other, and a tissue-engaging tip extends from a free end of each of the legs in a direction away from the other of the legs. In some embodiments, the biasing section biases the legs of the tissue traction device towards each other.

In some embodiments, the tissue-engaging tips of the tissue traction device are positioned within a seat defined in the delivery shaft.

In some embodiments, the tissue traction system includes a pusher configured to push the tissue traction device out of the delivery shaft. In some embodiments, the pusher is separably coupled with the tissue traction device.

In some embodiments, the tissue traction system includes a plurality of tissue traction devices maintained in delivery configurations within the lumen of the delivery shaft for delivery to a treatment site.

In some embodiments, the tissue traction system includes an endoscope having a working channel, the delivery shaft configured for delivery to a treatment site through the working channel of the endoscope.

In accordance with various principles of the present disclosure, a tissue traction device includes a pair of legs biased with respect to each other by a biasing section, the tissue traction device being shiftable between a delivery configuration in which the tissue traction device is deliverable through a lumen of a delivery shaft, and a deployment configuration in which the biasing section of the tissue traction device exerts traction on tissue to draw the tissue away from a treatment site; and at least one of the tissue traction device legs including a tissue-engaging tip configured to engage tissue and extending from a free end of the at least one of the legs and transverse to the at least one of the legs.

In some embodiments, the biasing section biases the legs of the tissue traction device away from each other, and the tissue-engaging tip extends from a free end of the at least one of the tissue traction device legs in a direction away from the other of the tissue traction device legs.

In some embodiments, the biasing section biases the legs of the tissue traction device towards each other, and the tissue-engaging tip extends from a free end of the at least one of the tissue traction device legs in a direction towards the other of the tissue traction device legs.

In accordance with various principles of the present disclosure, a method of applying traction to tissue to draw the tissue away from a treatment site to allow unimpeded access to the treatment site includes delivering a tissue traction device having a pair of legs biased with respect to each other by a biasing section to the treatment site; engaging a tissue-engaging portion extending from one of the pair of legs of the tissue traction device with tissue to be drawn away from the treatment site; engaging a tissue-engaging portion extending from the other of the pair of legs with tissue separate from the tissue to be drawn away from the treatment site; and exerting a biasing force from the biasing section with respect to the legs of the tissue traction device to apply traction to the tissue to be drawn away from the treatment site.

In some aspects, the method includes engaging a tissue-engaging portion of the other of the pair of legs comprises engaging the tissue-engaging portion of the other of the pair of legs with tissue at the treatment site and cut with respect to the tissue to be drawn away from the treatment site; and exerting a biasing force comprises expanding the legs apart from each other via the biasing section.

In some aspects, the method includes engaging a tissue-engaging portion of the other of the pair of legs comprises engaging the tissue-engaging portion of the other of the pair of legs with tissue spaced apart from the treatment site; and exerting a biasing force comprises drawing the legs toward each other via the biasing section.

In some aspects, engaging a tissue-engaging portion of at least one of the legs with tissue includes engaging the tissue-engaging portion with tissue in a direction in which the biasing section exerts a biasing force with respect to the leg from which the tissue-engaging portion extends.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1A illustrates a perspective view of an example of an embodiment of a tissue traction system in accordance with various principles of the present disclosure with a portion of a delivery shaft thereof in cross-section to show an example of an embodiment of a tissue traction device therein.

FIG. 1B illustrates a perspective view similar to that of FIG. 1A, but with the tissue traction device advanced to engage tissue.

FIG. 1C illustrates a system similar to the system illustrated in FIG. 1A and FIG. 1B being withdrawn from a treatment site and leaving a tissue traction device applying traction to tissue at the treatment site.

FIG. 2 illustrates an enlarged isolated cross-sectional view of the distal end of an example of an embodiment of a delivery shaft of a tissue traction system formed in accordance with various principles of the present disclosure with an example of an embodiment of a tissue traction device engaged therein.

FIG. 3 illustrates a perspective view of a distal end of an example of an embodiment of a delivery shaft of a tissue traction system such as illustrated in FIG. 2 with an example of an embodiment of a tissue traction device positioned therein.

FIG. 4 illustrates a perspective view of a distal end of an example of an embodiment of a delivery shaft of a tissue traction system such as illustrated in FIG. 2 but without the tissue traction device.

FIG. 5 illustrates an example of an embodiment of a tissue traction device formed in accordance with various principles of the present disclosure.

FIG. 6 illustrates an example of an embodiment of a tissue traction device formed in accordance with various principles of the present disclosure.

FIG. 7 illustrates an example of an embodiment of a tissue traction device formed in accordance with various principles of the present disclosure.

FIG. 8 illustrates an example of an embodiment of a tissue traction device formed in accordance with various principles of the present disclosure.

FIG. 9A-9F illustrate various stages of use of a tissue traction device and system in accordance with various principles of the present disclosure.

FIGS. 10A-10D illustrate an example of an embodiment of a tissue traction device and system and use thereof in various stages of deployment.

FIG. 11A illustrates another example of an embodiment of a tissue traction device formed in accordance with various principles of the present disclosure.

FIG. 11B illustrates a tissue traction device as illustrated in FIG. 11A, but in a different configuration.

FIGS. 12A-12D illustrate various stages of use of a tissue traction device and system such as illustrated in FIG. 11A and FIG. 11B in accordance with various principles of the present disclosure.

FIG. 13 illustrates another example of an embodiment of a tissue traction system in accordance with various principles of the present disclosure.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “free end” of an element is a terminal end at which such element does not extend beyond.

In accordance with various principles of the present disclosure, a tissue traction system and a tissue traction device are configured and designed to apply a traction force to tissue at a treatment site. It will be appreciated that terms such as traction, tension, retraction, separation, pulling, etc., including other grammatical forms thereof, may be used interchangeably herein without intent to limit. Further, it will be appreciated that the treatment site may be referenced alternatively herein as an anatomical site, target site, traction site, resection site, dissection site, etc., without intent to limit. In some embodiments, a tissue traction device formed in accordance with various principles of the present disclosure is deployed to separate a segment of tissue from adjacent and/or surrounding tissue. In some embodiments, tissue at the treatment site is cut, and the tissue traction device is positioned with respect to the cut tissue to separate (e.g., spread apart) the cut edges of tissue. Further procedures may then be performed at the treatment site, such as tissue dissection or tissue resection, with a tissue traction device formed in accordance with various principles of the present disclosure lifting a portion of tissue away from the treatment site. For instance, endoscopic submucosal dissection may be more readily performed with a tissue traction device lifting mucosal tissue away from submucosal tissue to facilitate treatment (e.g., dissection) of the submucosal tissue. For the sake of convenience and without intent to limit, the separated segment of tissue may be referenced herein as a tissue flap. It will be appreciated that, unless otherwise stated, reference to “at” the treatment site is intended to include tissue at and about the vicinity of (e.g., along, adjacent, etc.) the treatment site, and is not limited to tissue which is precisely at the location being treated. It will be appreciated that terms such as engage (and other grammatical forms thereof) may be used interchangeably herein with terms such as couple, grasp, hold, clasp, clip, anchor, attach, affix, secure, etc. (and other grammatical forms thereof), without intent to limit. It will be appreciated that terms such as portion, area, section, segment, etc., may be used interchangeably herein without intent to limit.

In some embodiments, the tissue traction device includes a first leg and a second leg joined together by a biasing section of the tissue traction device. The biasing section may bias the legs together or apart, selection of the direction of bias depending on the intended deployment of the tissue traction device. For instance, in some embodiments, a first of the legs of the tissue traction device may be engaged with tissue at a treatment site, and a second of the legs of the tissue traction device may be engaged with tissue separated from the treatment site (e.g., cut with respect to the treatment site), with the biasing section biasing the first and second legs apart from each other. Upon deployment of such embodiment, the legs move the tissue separated from the treatment site away from the tissue at the treatment site to facilitate access to the treatment site. As such, tissue cut away from the treatment site may form a flap which is moved away from the treatment site (e.g., lifted) by the second leg of the tissue traction device, with the first leg engaging tissue at or adjacent the treatment site. In other embodiments, a first of the legs of the tissue traction device may be engaged with tissue cut with respect to a treatment site (e.g., creating a tissue flap), and a second of the legs of the tissue traction device may be engaged with tissue spaced apart from the treatment site (e.g., distal to the treatment site), with the biasing section biasing the first and second legs together. Upon deployment of such embodiment, the legs move the tissue separated from the treatment site away from the treatment site towards the tissue spaced apart from the treatment site to facilitate access to the treatment site.

The legs of a tissue traction device formed in accordance with various principles of the present disclosure are coupled together via the biasing section and have free ends extending away from the coupled ends. The free ends of the tissue traction device legs may have tissue-engaging tips. For instance, in some embodiments, the tissue-engaging tips are thin or sharp or pointed or otherwise configure to penetrate tissue. In some embodiments, the tissue-engaging tips extend transverse to the extent of the legs from the biasing section to the free ends thereof up to the tissue-engaging tips. The tissue-engaging tips may extend in a direction with respect to the extent of the tissue traction device legs to facilitate engagement with tissue to apply traction to the tissue. For instance, the direction in which the tissue-engaging tips extend with respect to the tissue traction device legs may be based on the bias of the legs and/or the manner in which the tissue traction device is to apply traction to tissue (e.g., separating two areas of tissue such as a cut section of tissue away from tissue at a treatment site, or drawing a cut area of tissue away from a treatment site and towards tissue spaced apart from the treatment site).

The tissue traction system may include a delivery shaft in which one or more tissue traction devices formed in accordance with various principles of the present disclosure may be delivered to a treatment site. The system may also include one or more of: a pusher (e.g., for deploying one or more tissue traction devices out of a delivery shaft); a treatment tool (such as for performing a procedure with respect to the treatment site); an end effector (e.g., for manipulating tissue or components of the tissue traction system including, without limitation, the tissue traction device); tissue closure devices and/or systems (e.g., for closing tissue cut at the treatment site); and other additional devices and systems as may be appreciated by those of ordinary skill in the art as useful in a tissue traction system formed in accordance with various principles of the present disclosure.

Various embodiments of tissue traction devices, systems, and methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. The following description is of illustrative examples of embodiments only, and is not intended as limiting the broader aspects of the present disclosure.

In the accompanying drawings illustrating examples of embodiments of tissue traction devices and systems, it will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments with reference numbers greater than 100 are generally designated with the same reference numbers increased by a multiple of 100 and redundant description is generally omitted for the sake of brevity. Moreover, certain features in one embodiment may be used across different embodiments and are not necessarily individually labeled when appearing in different embodiments.

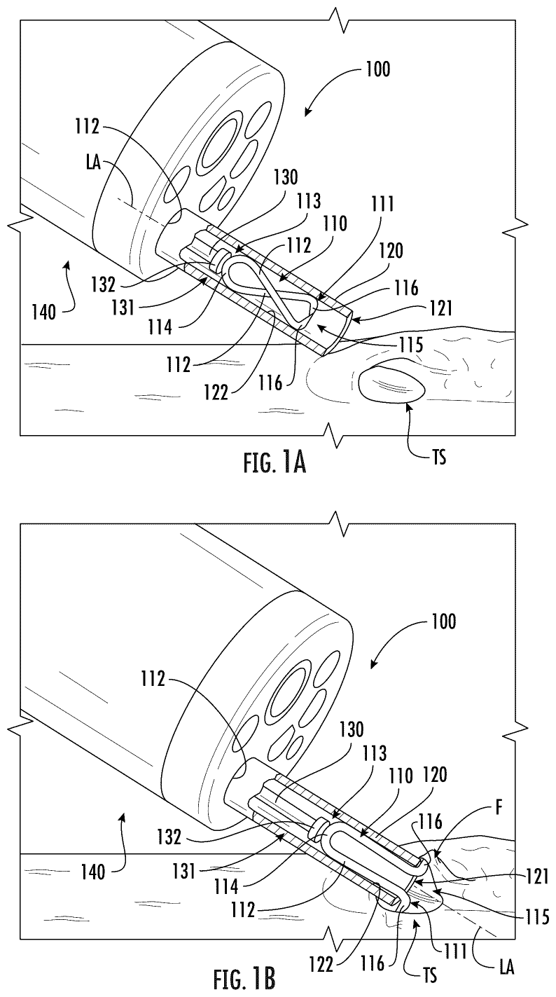

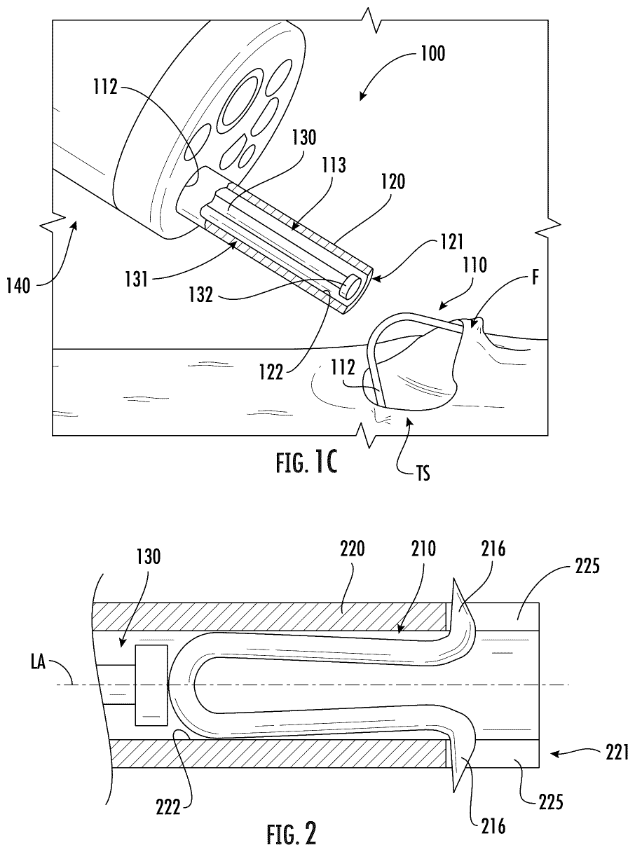

Turning now to the drawings, an example of an embodiment of a tissue traction system 100 is illustrated in FIGS. 1A-1C in various stages of delivery and deployment of a tissue traction device 110. The tissue traction device 110 may in some aspects be considered a part of the tissue traction system 100. The tissue traction system 100 includes a delivery shaft 120 defining a lumen 122 in which the tissue traction device 110 may be delivered to a treatment site TS, as illustrated in FIG. 1A. A pusher 130 may movably extend through the lumen 122 of the delivery shaft 120 to push the tissue traction device 110 distally out of the delivery shaft 120 upon reaching a treatment site TS. In the illustrated example of an embodiment, the pusher 130 has a distal end 131 abutting the proximal end 113 of the tissue traction device 110. Optionally, the pusher 130 has a wider (in a direction transverse to the longitudinal axis LA of the tissue traction system 100) or expanded distal section 132 configured to abut the proximal end 113 of the tissue traction device 110. Alternatively, as described in further detail below, the pusher 130 may be reversibly coupled (e.g., detachably coupled) with the tissue traction device 110.

The tissue traction device 110 includes at least two legs 112 coupled together by/along a biasing section 114. The legs 112 may be separately formed and joined to a separately formed biasing section 114 to form an integrated tissue traction device 110, or the tissue traction device 110 may be formed from a unitary structure, such as a wire or other elongated bendable and resilient material, bent into the desired shape/configuration. One or more sections of the tissue traction device 110 may be formed of a shape-memory and/or heat formable material, such as nickel-titanium alloy (e.g., nitinol). At least the biasing section 114 of the tissue traction device 110 is elastically biased to bias the legs 112 of the tissue traction device 110 in a desired direction (depending on the ultimate use of the tissue traction device 110, as described in further detail below).

In an example of an embodiment, as illustrated in FIGS. 1A-1C, the tissue traction device 110 is delivered to a treatment site TS within the delivery shaft 120 in a constrained configuration in which the legs 112 must be held together against a biasing force of the biasing section 114 which biases or drives the legs 112 apart. The legs 112 may alternately be described as compressed, contracted, constrained, restrained, etc., without intent to limit.

Once the tissue traction system 100 is delivered to a treatment site TS, such as illustrated in FIG. 1A, the pusher 130 may be distally advanced to distally advance the tissue traction device 110 to engage tissue at the treatment site TS, such as illustrated in FIG. 1B. The pusher 130 may be advanced by controlling a proximal end thereof such as with a control handle, not shown but which may be in any form known to those of ordinary skill in the art, the configuration of which is not critical to and does not limit the present disclosure. Once the distal end 111 of the tissue traction device 110 is advanced beyond the distal end 121 of the delivery shaft 120, tissue-engaging tips 116 of the tissue traction device 110 may be engaged with tissue at or along the treatment site TS, as illustrated in FIG. 1B. In the example of an embodiment illustrated in FIGS. 1A-1C, the tissue-engaging tips 116 are formed along free ends 115 of the legs 112, however other positions and/or configurations are within the scope and spirit of the present disclosure. Additionally or alternatively, the tissue-engaging tips 116 extend transverse to the portion of the legs 112 from which the tissue-engaging tips 116 extend. For instance, the tissue-engaging tips 116 may extend transverse to the legs 112, such as transverse to the length of the legs 112 (defined generally along a direction in which the legs 112 extend from the biasing section 114 toward the distal end 111 of the tissue traction device 110 and/or along the longitudinal axis LA).

In some aspects, at least one of the tissue-engaging tips 116 extends generally in the same direction of the biasing force applied by the biasing section 114 to the legs 112. Such configuration may facilitate engagement and/or penetration of the tissue-engaging tips 116 with tissue. In other words, the tissue-engaging tip 116 of one leg 112 of the tissue traction device 110 may extend away from the other leg 112 if the legs 112 are biased away from each other by the biasing section 114. In the example of an embodiment illustrated in FIGS. 1A-1C, the legs 112 of the tissue traction device 110 are biased apart by the biasing section 114 of the tissue traction device 110 to separate sections of tissue to which the tissue-engaging tips 116 are respectively engaged. Accordingly, the tissue-engaging tips 116 may extend outwardly from a longitudinal axis LA of the tissue traction system 100/tissue traction device 110 when the tissue traction device 110 is engaged with tissue, as illustrated in FIG. 1B.

As the tissue traction device 110 is distally advanced out of the delivery shaft 120, the legs 112 are released from a constrained configuration within the lumen 122 of the delivery shaft 120 (such as illustrated in FIG. 1A and FIG. 1B), and begin to apply traction to tissue at and along the treatment site TS. For instance, once the distal end 111 of the tissue traction device 110 is extended past the distal end 121 of the delivery shaft 120, the free ends 115 of the legs 112 move to an unconstrained or expanded or neutral or traction-applying configuration (such terms being used interchangeably herein without intent to limit) with the legs 112 moving apart from each other (e.g., moving in a direction away from the longitudinal axis LA). When unconstrained, the legs 112 of the tissue traction device 110 may exert traction to tissue sections engaged thereby (e.g., by the free ends 115 thereof). As illustrated in FIG. 1B and FIG. 1C, one leg 112 of the tissue traction device 110 is engaged (e.g., at a tissue-engaging tip 116 thereof) with tissue at the treatment site TS, and another leg 112 of the tissue traction device 110 is engaged (e.g., at a tissue-engaging tip 116 thereof) with tissue separated from tissue at the treatment site TS by an incision or cut. As the legs 112 are unconstrained, and allowed to expand apart (e.g., by being biased apart by the biasing section 114), the legs 112 apply traction to the tissue cut with respect to the treatment site TS. The tissue cut with respect to the treatment site TS may be considered to form a flap F which is lifted away from the treatment site TS by the leg 112 opposite the leg 112 engaged with tissue at the treatment site TS, as illustrated in FIG. 1B and FIG. 1C. It will be appreciated that the tissue separated from the treatment site TS may be referenced herein as a flap F for the sake of convenience and without intent to limit. Once the tissue traction device 110 is fully deployed, as illustrated in FIG. 1C, the delivery shaft 120 may be retracted and the tissue traction device 110 left in place to clear the treatment site TS of the cut tissue (flap F). As may be appreciated, the lifting of the flap F with respect to the treatment site TS by the expanded tissue traction device 110 allows a further procedure (such as endoscopic submucosal resection of tissue at the treatment site TS and/or under the tissue being lifted) to be performed at the treatment site TS without interference of the flap F. For instance, a procedure may be performed under the flap F without such tissue impeding or otherwise interfering with access to the treatment site TS as may otherwise occur.

Optionally, the tissue traction system 100 is delivered through another delivery device or system, such as an endoscope 140. The delivery system 100 may be advanced distally through a working channel 142 of the endoscope 140 to the treatment site TS to deploy the tissue traction device 110 (e.g., as illustrated in FIG. 1A and FIG. 1B). Once the tissue traction device 110 has been deployed, the delivery shaft 120 may be retracted or withdrawn proximally through the working channel 142 of the endoscope 140 (e.g., as illustrated in FIG. 1C). Such advancement and withdrawal of the delivery shaft 120 may be performed in a manner known to those of ordinary skill in the art, and thus further details are not deemed to be necessary for full disclosure of the various principles of the present disclosure.

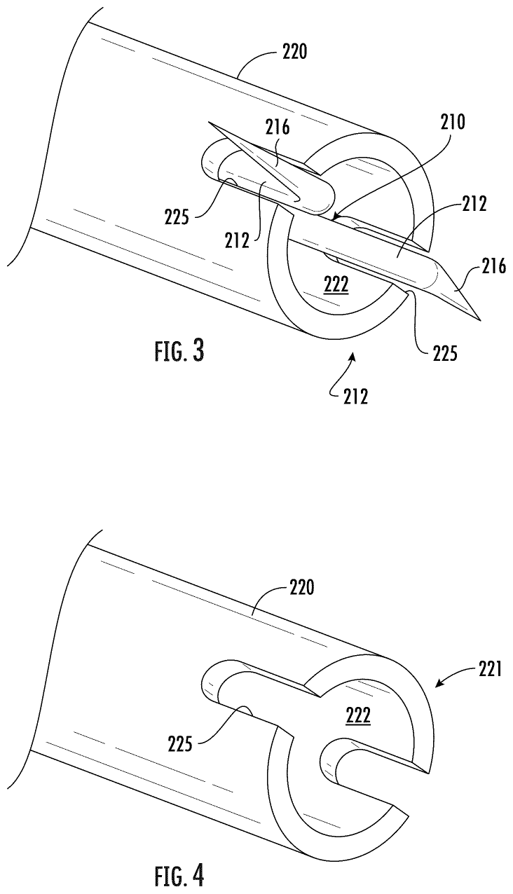

In some embodiments, the tissue traction device 110 may be delivered substantially fully within the delivery shaft 120, such as with the distal end 111 of the tissue traction device 110 within the delivery shaft 120 and proximal to the distal end 121 of the delivery shaft 120, as illustrated in FIG. 1A. However, in other embodiments, a portion of the distal end of the tissue traction device may engage and/or extend outwardly from the delivery shaft during delivery thereof. For instance, as illustrated in FIGS. 2-4, a distal portion 210 of a tissue traction device 210, such as the tissue-engaging tips 216 of the legs 212 of the tissue traction device 210, may be positioned within a seat 225 defined within a section of the distal end 221 of a delivery shaft 220. In some embodiments, portions of the tissue traction device 210 may extend outwardly from the delivery shaft 220 during delivery thereof (e.g., away from the longitudinal axis LA of the system/device), such as illustrated in FIG. 2 and FIG. 3. The seat 225 may be formed within the lumen 222 of the delivery shaft 220 and optionally may extend through the wall of the delivery shaft 220 such as to form a slot, such as illustrated in FIG. 4.

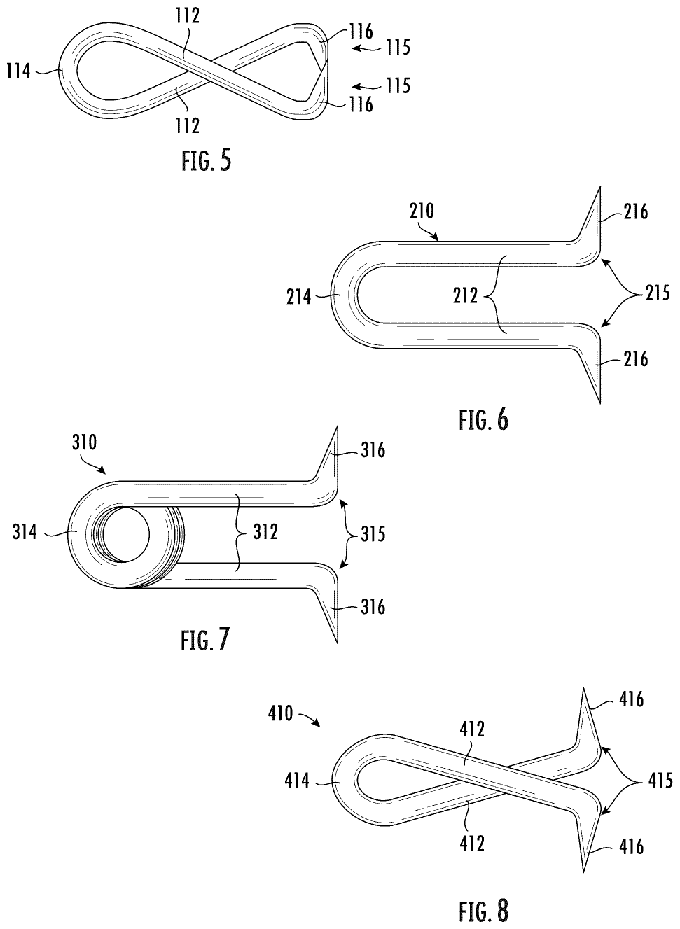

It will be appreciated that a tissue traction device formed in accordance with principles of the present disclosure may have any of a variety of configurations allowing the legs thereof to exert traction forces with respect to tissue sections. Various examples of configurations of tissue traction devices formed in accordance with various principles of the present disclosure are illustrated in FIGS. 5-7. It will be appreciated that other configurations of tissue traction device may be formed in accordance with various principles of the present disclosure, the present disclosure not being limited by the illustrated examples of embodiments.

The example of an embodiment of a tissue traction device 110 illustrated in FIG. 5 is similar to the example of an embodiment of a tissue traction device 110 illustrated in FIGS. 1A-1C and thus features thereof are indicated with the same reference numerals, and reference is made to the above description of various features thereof. The tissue traction device 110 illustrated in FIG. 5 may be used with a tissue traction system 100 in a manner as illustrated in FIGS. 1A-1C. In an embodiment in which the tissue traction device 110 is configured to separate tissue, such as for use as illustrated in FIGS. 1A-1C, the legs 112 of the tissue traction device 110 illustrated in FIG. 5 may be considered to be illustrated in a constrained configuration, generally crossing each other. The legs 112 are subject to a biasing force exerted by the biasing section 114. When the legs 112 are unconstrained, the legs 112 (particularly, the free ends 115 thereof) are biased apart by the biasing section 114 into an expanded configuration, such as into the configuration illustrated in FIG. 1C. The tissue-engaging tips 116 of the legs 112 of the tissue traction device 110 of FIG. 5 may extend outwardly away from each other to engage tissue sections to be spaced apart from each other by the expanded tissue traction device 110.

The example of an embodiment of a tissue traction device 210 illustrated in FIG. 6 is similar to the example of an embodiment of a tissue traction device 110 illustrated in FIG. 5. However, in contrast with the example of an embodiment of a tissue traction device 110 illustrated in FIG. 5, the free ends 215 of the legs 212 of the tissue traction device 210 illustrated in FIG. 6 are spaced apart, or at least do not cross each other. As such, the tissue traction device 210 may be considered similar to the example of an embodiment of a tissue traction device 210 illustrated in FIGS. 2-4. Thus, features of the tissue traction device 210 illustrated in FIG. 6 are indicated with the same reference numerals as the tissue traction device 210 illustrated in FIGS. 2-4, and reference is made to the above description of various features thereof. It will be appreciated that if the tissue traction device 210 is configured to separate tissue, then the tissue traction device 210 may be considered to be illustrated in FIG. 6 in a constrained configuration. The biasing section 214 nonetheless exerts sufficient force with respect to the legs 212 to further bias the legs 212 apart into an expanded configuration such as illustrated in FIG. 1C.

The example of an embodiment of a tissue traction device 310 illustrated in FIG. 7 is similar to the example of an embodiment of a tissue traction device 210 illustrated in FIG. 6 in that the legs 312 do not cross and/or the free ends 315 thereof may be spaced apart. However, the biasing section 314 of the tissue traction device 310 illustrated in FIG. 7 is coiled, such as to provide increased biasing force on the legs 312 of the tissue traction device 310. If the tissue traction device 310 is configured to separate tissue, then the tissue traction device 310 may be considered to be illustrated in a constrained configuration and the biasing section 314 biases the legs 312 apart into an expanded configuration such as illustrated in FIG. 1C.

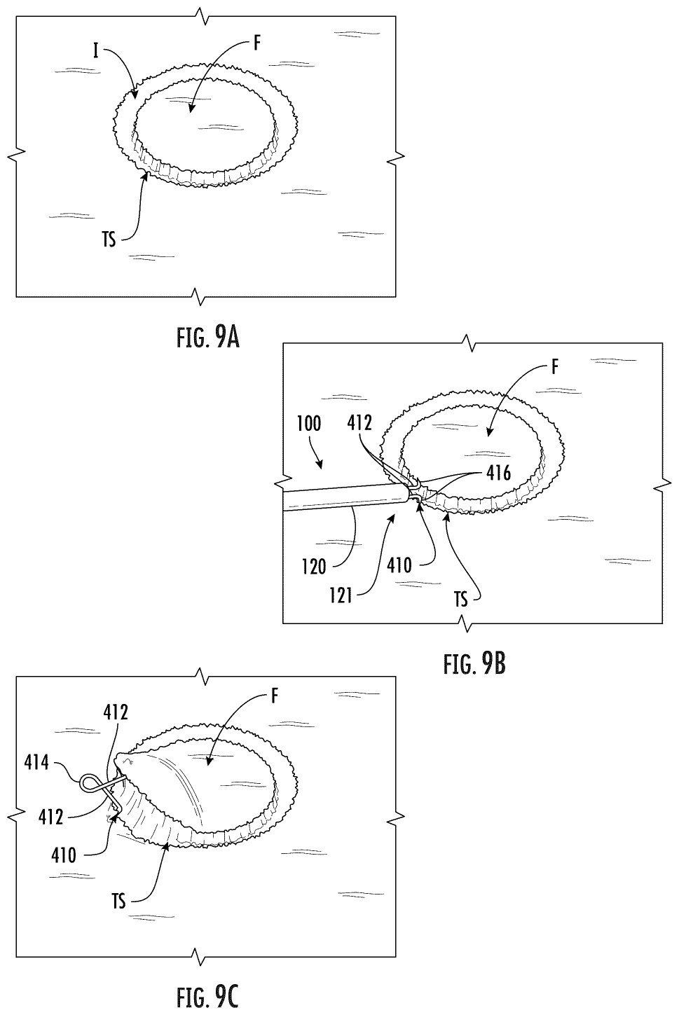

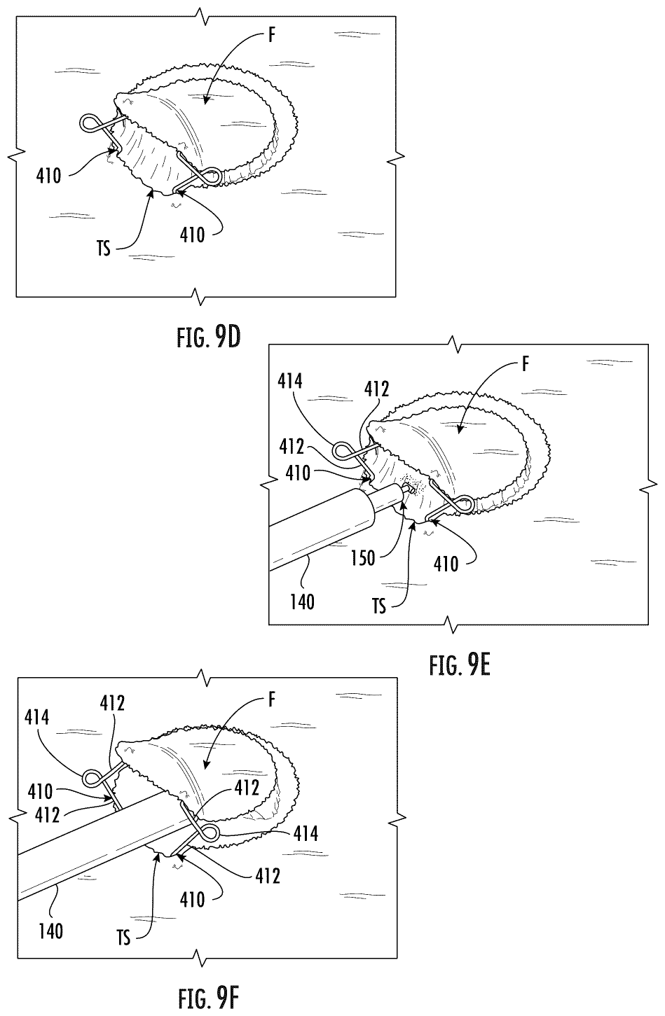

Like the example of an embodiment of a tissue traction device 110 illustrated in FIG. 5, the legs 412 of the example of an embodiment of a tissue traction device 410 illustrated in FIG. 8 cross each other. However, if the tissue traction device 410 is configured to separate tissue, instead of the biasing section 414 biasing the legs 412 apart into an uncrossed configuration (as with the tissue traction device 110 of FIG. 5, such as in the example of an embodiment of a use thereof illustrated in FIG. 1B and FIG. 1C), the biasing section 414 biases the legs 412 into a further crossed configuration (as compared to the configuration shown in FIG. 8), such as illustrated in FIGS. 9C-9F (discussed in further detail below). It will be appreciated that if the tissue traction device 410 is configured to separate tissue, then the tissue traction device 410 may be considered to be illustrated in a constrained configuration in FIG. 8. Further, it will be appreciated that in embodiments such as those in which the tissue traction device 410 is configured to separate tissue, the tissue-engaging tips 416 may extend in a direction opposite the direction in which the tissue-engaging tips 116 of the tissue traction device 110 illustrated in FIG. 5 extend so that the tissue-engaging tips 416 extend into the tissue sections to be separated once the tissue traction device 410 expands into a traction-applying configuration. Various other orientations of tissue-engaging tips are within the scope and spirit of the present disclosure.

An example of a manner of using a tissue traction system 100, 200 and/or a tissue traction device 110, 210, 310, 410 as described above is illustrated in FIGS. 9A-9F. As illustrated in FIG. 9A, an incision I is made along a treatment site TS at which a procedure is to be performed. The incision I is illustrated as encircling the treatment site TS, but may be more limited than the illustrated extent (e.g., just a small incision rather than an incision around the entire perimeter of the treatment site TS, or even no incision at all). It will be appreciated that the incision I may formed in any desired manner known to those of ordinary skill in the art, the present disclosure not being limited in this regard. The incision I delineates/differentiates the treatment site TS from the tissue to be separated therefrom, hereinafter referenced as a flap F for the sake of convenience and without intent to limit.

To facilitate access to the treatment site TS, the tissue traction system 100 is delivered to the treatment site TS, as illustrated in FIG. 9B. A tissue traction device 410 such as illustrated in FIG. 8 is illustrated extended from the distal end 121 of the delivery shaft 120 with the tissue-engaging tips 416 of the respective legs 412 engaging the treatment site TS and the flap F, respectively. It will be appreciated that any of the other tissue traction devices 110, 210, 310, or other configurations of tissue traction devices formed in accordance with various principles of the present disclosure may be used instead, the tissue traction device 410 being illustrated for the sake of convenience and without intent to limit.

As illustrated in FIG. 9C, once the tissue traction device 410 is delivered (fully extended outside the delivery shaft 120 illustrated in FIG. 8B), the legs 412 are biased apart by the biasing section 414 to separate the flap F from the treatment site TS to allow access to the treatment site TS and/or to tissue below the flap F. Additional tissue traction devices, such as tissue traction device 410 or other tissue traction devices formed in accordance with various principles of the present disclosure, may be used to apply further traction to the flap F to separate the flap F from the treatment site TS, such as illustrated in FIG. 9D.

Once the flap F is sufficiently distanced from the treatment site TS, a treatment tool 150 (e.g., cutting device such as a blade, cauterizer, irrigation device, suction device, needle for delivery of one or more injectable treatment materials, etc.) may be delivered (e.g., with the use of an endoscope 140) to perform a procedure at the treatment site TS, as illustrated in FIG. 9E. As may be appreciated with reference to FIG. 9F, the tissue traction devices 410 provides sufficient traction on the flap F to allow unimpeded access for the treatment tool 150 for a medical professional to perform the desired procedure with respect to the treatment site TS.

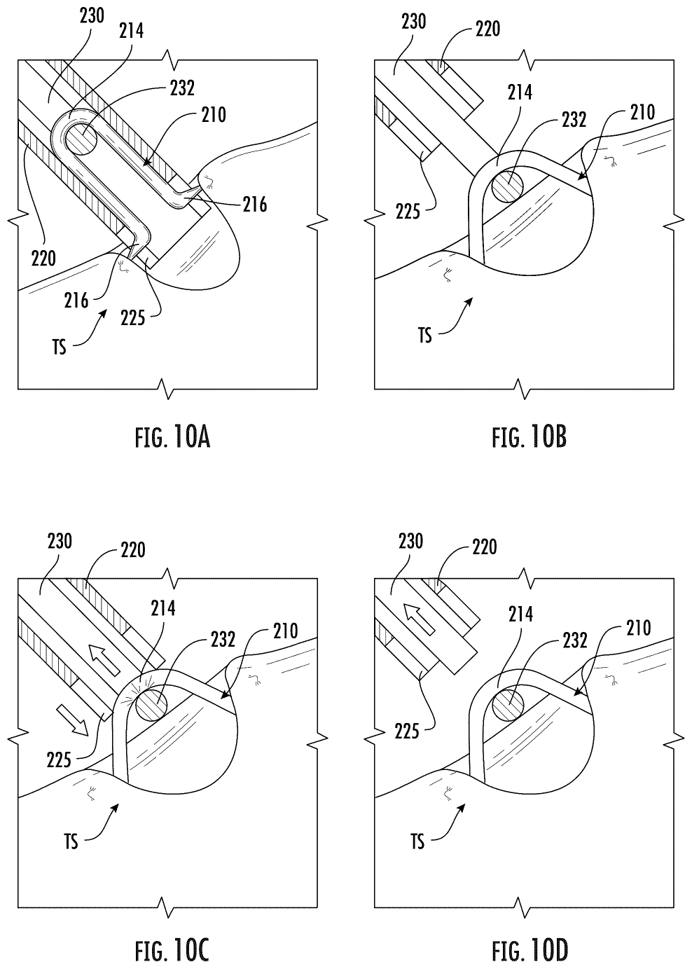

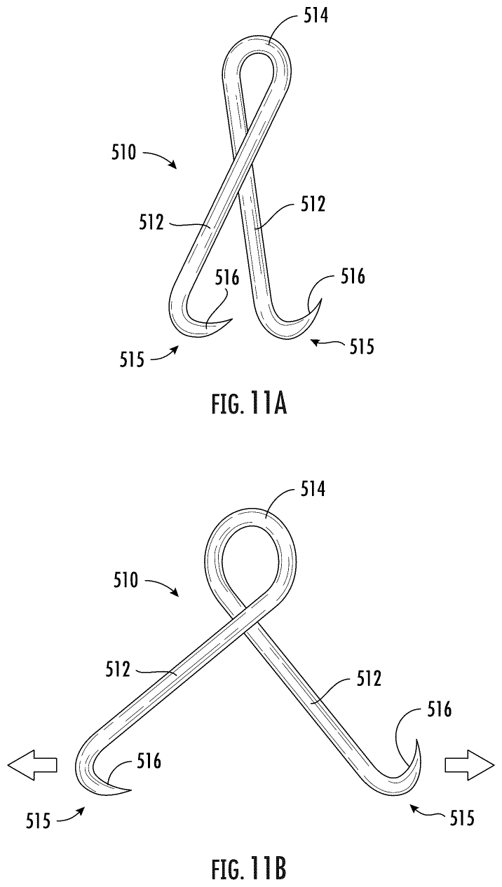

It will be appreciated that various modifications to various components or aspects or actions described above may be made without departing from the spirit and scope of the present disclosure. For instance, as noted above, a pusher 130 may be formed to abut a tissue traction device 110 formed in accordance with various principles of the present disclosure or may be coupled with a tissue traction device 110 formed in accordance with various principles of the present disclosure. An example of an embodiment of a pusher 230 configured to be separably coupled with an example of an embodiment of a tissue traction device 210, and manner of use thereof, are illustrated in FIGS. 10A-10D. It will be appreciated that a tissue traction device 210 such as illustrated in FIG. 6 and a delivery shaft 220 such as illustrated in FIGS. 2-4 are illustrated in the example of an embodiment illustrated in FIGS. 10A-10D. However, other tissue traction devices and delivery shafts as described herein or otherwise formed in accordance with various principles of the present disclosure may be used instead.

As illustrated in FIG. 10A, the pusher 230 is coupled to the tissue traction device 210 via a separable distal section 232. In the illustrated example of an embodiment, the separable distal section 232 is coupled with the biasing section 214 of the tissue traction device 210. However, the separable distal section 232 may be coupled with other sections/portions of the tissue traction device 210 instead. The separable distal section 232 may be looped or hooked or welded with respect to (e.g., around) a portion of the tissue traction device 210 in any desired appropriate manner as may be appreciated by those of ordinary skill in the art to allow the pusher 230 to advance the tissue traction device 210 as desired while also allowing separation therefrom as described below. For instance, the separable distal section 232 may be separated from the tissue traction device 210, such as disconnecting (e.g., breaking) therefrom. Additionally or alternatively, deformation of the separable distal section 232 allows release of the separable distal section 232 from the tissue traction device 210.

Once the tissue-engaging tips 216 of the tissue traction device 210 have been engaged with tissue at the treatment site TS, as illustrated in FIG. 10A, one or both of the delivery shaft 220 and the pusher 230 may be moved, as illustrated in FIG. 10C, to separate the pusher 230 from the tissue traction device 210. For instance, the delivery shaft 220 may be advanced distally towards the tissue traction device 210 to push the tissue traction device 210 away from the pusher 230, such as to disconnect the pusher 230 from the tissue traction device 210. Additionally or alternatively, the pusher 230 may be pulled proximally to be retracted away from the tissue traction device 210 to be disconnected therefrom. Movements causing the tissue traction device 210 to be moved away from the pusher 230, such as in the direction of the arrows illustrated in FIG. 10C, apply tension in/to the pusher 230, particularly with respect to a frangible section just proximal to the separable distal section 232. Once sufficient tension has been applied, the separable distal section 232 of the pusher 230 separates (e.g., breaks off) from the portion of the pusher 230 proximal thereto to deploy the tissue traction device 210, as illustrated in FIG. 10D.

It will be appreciated that other configurations of separable sections of a pusher are within the scope and spirit of the present disclosure. For instance, the separable distal section of the pusher may be configured to extend through an aperture through the tissue traction device, such as through the biasing section thereof. The separable distal section may have a geometry that is selectively movable between an engaged and a disengaged configuration with respect to the tissue traction device. For instance, the separable distal section and the tissue traction device may have a key and keyhole type arrangement such as known to those of ordinary skill in the art. For instance, the separable distal section of the pusher may be movable with respect to the tissue traction device, such as to be insertable within an aperture within a portion of the tissue traction device. The separable distal section of the pusher may be moved with respect to the tissue traction device to prevent relative movement therebetween. For instance, the shape of the separable distal section (e.g., key) may align with a shape of the aperture (e.g., keyhole) in the tissue traction device to allow the separable distal section to pass through the aperture in the tissue traction device. The separable distal section may then be advanced past the aperture in the tissue traction device and rotated out of alignment with the aperture to prevent withdrawal of the pusher with respect to the tissue traction device. To deploy the tissue traction device, the pusher may be rotated or otherwise moved/manipulated with respect to the tissue traction device so that the geometry of the separable distal section of the pusher matches or is otherwise aligned with the aperture in the tissue traction device for withdrawal and separation therefrom. For instance, the separable distal section may be elongated, the aperture in the tissue traction device may be elongated, and the pusher may be rotatable to move the separable distal section thereof alignment with the aperture to pass through and distal to the aperture. Once the separable distal section of the pusher has moved past the aperture in the tissue traction device, the pusher may be moved or rotated so that the separable distal section is out of alignment with the aperture to interlock, engage, couple, etc., the pusher with the tissue traction device. Various and numerous configurations embodying such concepts may be appreciated by those of ordinary skill in the art and are not enumerated herein or illustrated in the accompanying drawings for the sake of brevity.

Other variations to the above-described tissue traction device, system, and method may be with regard to the direction of traction applied to tissue (such as to tissue to be separated from a treatment site TS). Additionally or alternatively, other variations to the above-described tissue traction device, system, and method may be with regard to the biasing force to be applied to the legs of a tissue traction device formed in accordance with various principles of the present disclosure. Additionally or alternatively, other variations to the above-described tissue traction device, system, and method may be with regard to the manner in which the legs of the tissue traction device engages tissue. Various other modifications may be made without departing from the scope and spirit of the principles of the present disclosure.

For instance, as noted above, a tissue traction device formed in accordance with various principles of the present disclosure may be configured so that the legs thereof are in a naturally closed configuration when deployed. In such embodiment, instead of the tissue-engaging portions of the tissue traction device expanding and separating apart from each other, the tissue-engaging portions are biased to be brought or drawn closer together. The legs of such tissue traction device may thus draw tissue together, such as to pull tissue away from a treatment site TS and towards an anchoring tissue site, thereby lifting the tissue (e.g., creating and lifting a flap of tissue) away from the treatment site. Various modifications to the above-described tissue traction devices may be made to form such embodiment. One example of an embodiment of a tissue traction device 510 with legs 512 configured to draw tissue together is illustrated in FIG. 11A and FIG. 11B.

The example of an embodiment of a tissue traction device 510 illustrated in FIG. 11A and FIG. 11B is similar to the tissue traction devices 110 and 410 illustrated in FIG. 5 and FIG. 8 and described above in that the legs 512 thereof cross each other. However, unlike the biasing sections 114 and 414 of the tissue traction devices 110, 410 (which biases the legs 112, 412 apart), the biasing section 514 of the tissue traction device 510 illustrated in in FIG. 11A and FIG. 11B draws the legs 512 closer together. Thus, the tissue traction device 510 may be considered to be illustrated in a neutral or relaxed configuration in FIG. 11A, and in an expanded, traction-applying configuration in FIG. 11B. More particularly, the legs 512 of the tissue traction device 510 may be considered to be illustrated in FIG. 11B as being pulled apart from each other in the direction of the illustrated arrows. The biasing section 514 exerts a biasing forced against the legs 512 of the tissue traction device 510 in the configuration illustrated in FIG. 11B to draw the legs 512 back together to the configuration illustrated in FIG. 11A. The legs 512 may thus be coupled to different sections of tissue spaced apart from each other to draw such sections together. For instance, the legs 512 may be coupled to cut tissue and an anchoring tissue spaced apart from the cut tissue to move the cut tissue (e.g., as a tissue flap) from a treatment site so that a procedure may be performed at the treatment site, such as under the cut tissue without interference of the cut tissue.

Another difference between the tissue traction device 510 illustrated in FIGS. 11A and 11B is that the tissue-engaging tips 516 thereof may be configured differently to correspond with the different direction in which a biasing force or traction is applied by the biasing section 514 to the legs 512 and/or by the legs 512 to tissue engaged by the tissue traction device 510. For instance, if the legs 512 are biased towards each other, then the tissue-engaging tips 516 may be arranged differently from the tissue-engaging tips of the tissue traction devices described above. For instance, in the example of an embodiment illustrated in FIG. 11A and FIG. 11B, the tissue-engaging tip 516 of at least the leg 512 which engages the tissue flap (tissue separated from a treatment site) is directed toward the other leg 512 when the tissue traction device 510 is in a neutral configuration, such as illustrated in FIG. 11A. The other leg 512 of the tissue traction device 510 may be engaged with tissue which may be considered anchoring tissue towards which the flap is drawn. The leg 512 engaging the anchoring tissue may have a tissue-engaging tip 516 extending away from the leg 512 engaged with the tissue flap, as illustrated, or, in some embodiments, extending toward the leg 512 engaged with the tissue flap. It will be appreciated that configurations of the tissue-engaging tips 516 of a tissue traction device 510 with legs 512 biased towards each other may be determined or formed based on the ultimate use of the tissue traction device 510.

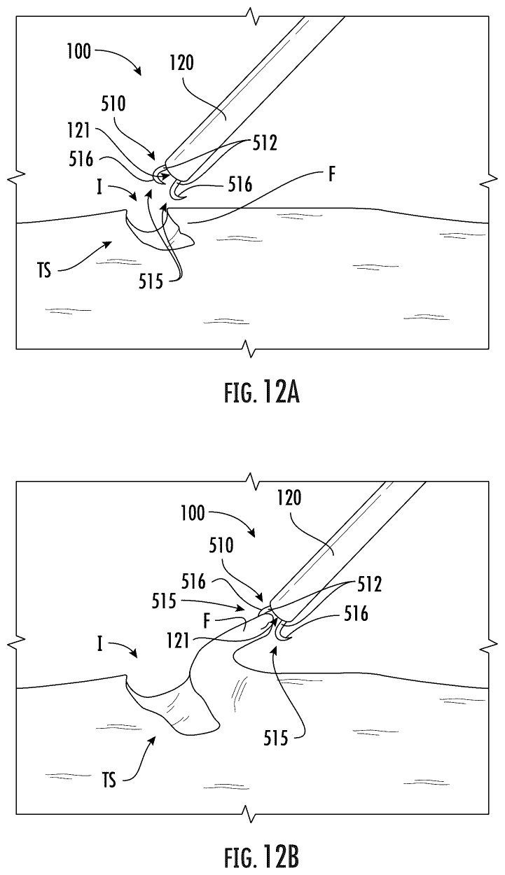

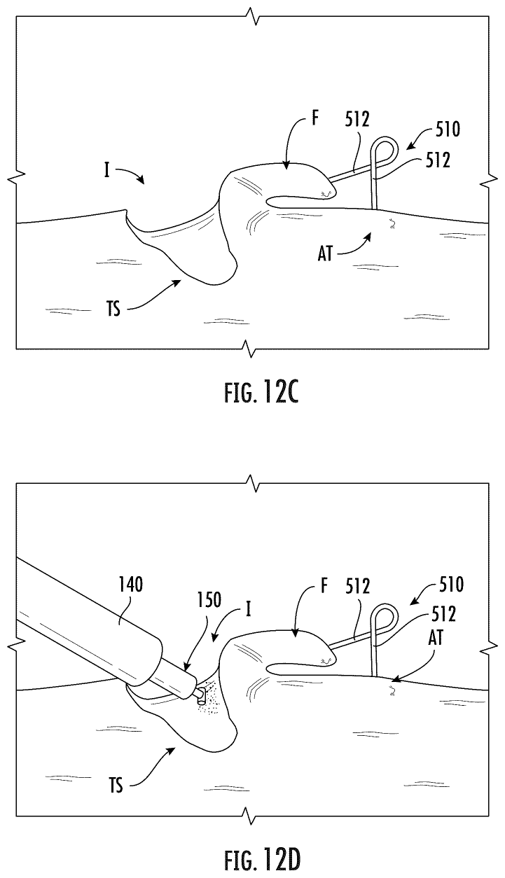

An example of a manner of using a tissue traction device 510 such as illustrated in FIG. 11A and FIG. 11B is illustrated in FIGS. 12A-12D. As illustrated in FIG. 12A, an incision I is made at a treatment site TS at which a procedure is to be performed. It will be appreciated that the incision I may formed in any desired manner known to those of ordinary skill in the art, the present disclosure not being limited in this regard. The incision I delineates/differentiates the treatment site TS from the tissue to be separated therefrom, hereinafter referenced as a flap F for the sake of convenience and without intent to limit. To facilitate access to the treatment site TS, a tissue traction system 100 is delivered to the treatment site TS, as illustrated in FIG. 12A. The free ends 515 of the legs 512 of a tissue traction device 510 such as illustrated in FIG. 11A and FIG. 11B are illustrated as extended distally from the distal end 121 of the delivery shaft 120 so that the tissue-engaging tips 516 of the respective legs 512 may be engaged with tissue.

In FIG. 12B, a tissue-engaging tip 516 of one of the legs 512 is engaged with tissue separated from the treatment site TS by the incision I. For the sake of convenience, and without intent to limit, such tissue may be referenced herein as a flap F and such leg 512 may be referenced herein as a flap-engaging leg 512. The tissue traction system 100 may then be moved away from the treatment site TS, pulling the tissue traction device 510 and the flap F away from the treatment site TS to allow unimpeded access to the treatment site TS, such as to perform a procedure at the treatment site TS.

As illustrated in FIG. 12C, the other leg 515 of the tissue traction device 510 may be engaged with tissue spaced apart from the treatment site TS. Such tissue may act to anchor the tissue traction device 510 to exert traction on the flap F and may thus be referenced herein as anchoring tissue AT for the sake of convenience and without intent to limit. Because the legs 512 of the tissue traction device 510 are biased together by the biasing section 514, once both of the legs 512 of the tissue traction device 510 are engaged with tissue, the biasing section 514 draws the legs 512 together to pull the flap F away from the treatment site TS to allow access to the treatment site TS and/or to tissue below the flap F. Additional tissue traction devices may be used to apply further traction to the flap F to separate the flap F from the treatment site TS.

Once the flap F is sufficiently distanced from the treatment site TS, a treatment tool 150 (e.g., such as described above) may be delivered (e.g., with the use of an endoscope 140) to perform a procedure at the treatment site TS, as illustrated in FIG. 12D. As may be appreciated, the tissue traction devices 510 provides sufficient traction on the flap F to allow unimpeded access for the treatment tool 150 for a medical professional to perform the desired procedure with respect to the treatment site TS.

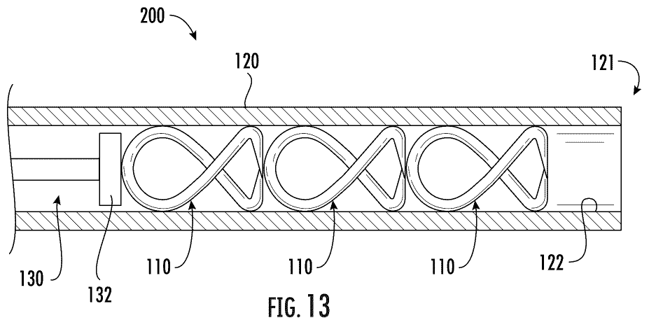

In addition to variations to the tissue traction device, variations to the tissue traction system described above may be made without departing from the scope and spirit of the present disclosure. For instance, a tissue traction system 200 such as illustrated in FIG. 13 may deliver more than one tissue traction device, such as a tissue traction device 110 formed in accordance with various principles of the present disclosure. As illustrated in FIG. 13, more than one tissue traction device 110 may be loaded within the lumen 122 of the delivery shaft 120 of the illustrated tissue traction system 200. For instance, two or more tissue traction devices 110 may be pre-loaded into the delivery shaft 120, such as lined up in series, end to end, stacked, etc. In some embodiments, the tissue traction devices 110 can push one another to allow for deployment. A pusher 130 such as described above or in any other configuration known to those of ordinary skill in the art may be used to push the tissue traction devices 110, such as the proximal-most tissue traction device 110 to push the more distal tissue traction devices 110 out of the delivery shaft 120.

It will be appreciated that various principles of the present disclosure may be applied in other manners. For instance, a system and/or device formed in accordance with various principles of the present disclosure can be used for other applications, such as keeping a lumen expanded whenever is needed, or for maintaining a tunnel open such as in procedures such as G-POEM (gastro-intestinal peroroal endoscopic myotomy), or maintaining intramural or submucosal space, or for other procedures benefiting from one or more principles of the present disclosure.

It is to be understood by one of ordinary skill in the art that the present discussion is a description of illustrative examples of embodiments only, and is not intended as limiting the broader aspects of the present disclosure.

All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

It will be appreciated that various features described with respect to one embodiment may be applied to another embodiment, whether or not explicitly indicated. The various features hereinafter described may be used singly or in any combination thereof. Therefore, the present invention is not limited to only the embodiments specifically described herein. Moreover, it will be appreciated that various further benefits of the various aspects, features, components, and structures of a tissue traction device and system as described herein, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements, components, features, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

1-15. (canceled)

16. A tissue traction system comprising:
a tissue traction device comprising a pair of legs biased with respect to each other by a biasing section; and
a delivery shaft defining a lumen within which said tissue traction device is deliverable to a treatment site;
wherein:
said tissue traction device is shiftable between a delivery configuration and a deployment configuration;
said tissue traction device legs include tissue-engaging tips configured to engage tissue;
said delivery shaft is configured to maintain said tissue traction device in the delivery configuration; and
in the deployment configuration, said biasing section of said tissue traction device exerts traction on tissue through said tissue traction device legs to draw tissue away from a treatment site.

17. The tissue traction system of claim 16, wherein said biasing section joins said legs of said tissue traction device together.

18. The tissue traction system of claim 16, wherein said biasing section biases said legs of said tissue traction device away from each other to lift tissue away from a treatment site.

19. The tissue traction system of claim 16, wherein said biasing section biases said legs of said tissue traction device towards each other to lift tissue away from a treatment site and towards anchoring tissue spaced apart from the treatment site.

20. The tissue traction system of claim 16, wherein at least one of said tissue-engaging tips extends from one of said legs transverse to said one of said legs and in a direction in which said biasing section biases said one of said legs.

21. The tissue traction system of claim 20, wherein said at least one of said tissue-engaging tip extends from a free end of said one of said legs.

22. The tissue traction system of claim 21, wherein said biasing section biases said legs of said tissue traction device away from each other, and a tissue-engaging tip extends from a free end of each of said legs in a direction away from the other of said legs.

23. The tissue traction system of claim 21, wherein said biasing section biases said legs of said tissue traction device towards each other.

24. The tissue traction system of claim 16, wherein said tissue-engaging tips of said tissue traction device are positioned within a seat defined in said delivery shaft.

25. The tissue traction system of claim 16, further comprising a pusher configured to push said tissue traction device out of said delivery shaft.

26. The tissue traction system of claim 25, wherein said pusher is separably coupled with said tissue traction device.

27. The tissue traction system of claim 16, further comprising a plurality of tissue traction devices maintained in delivery configurations within the lumen of said delivery shaft for delivery to a treatment site.

28. The tissue traction system of claim 16, further comprising an endoscope having a working channel, said delivery shaft configured for delivery to a treatment site through the working channel of said endoscope.

29. A tissue traction device comprising a pair of legs biased with respect to each other by a biasing section, wherein:
said tissue traction device is shiftable between a delivery configuration in which said tissue traction device is deliverable through a lumen of a delivery shaft, and a deployment configuration in which said biasing section of said tissue traction device exerts traction on tissue to draw the tissue away from a treatment site; and
at least one of said tissue traction device legs includes a tissue-engaging tip configured to engage tissue and extending from a free end of said at least one of said legs and transverse to said at least one of said legs.

30. The tissue traction system of claim 29, wherein said biasing section biases said legs of said tissue traction device away from each other, and said tissue-engaging tip extends from a free end of said at least one of said tissue traction device legs in a direction away from the other of said tissue traction device legs.

31. The tissue traction system of claim 29, wherein said biasing section biases said legs of said tissue traction device towards each other, and said tissue-engaging tip extends from a free end of said at least one of said tissue traction device legs in a direction towards the other of said tissue traction device legs.

32. A method of applying traction to tissue to draw the tissue away from a treatment site to allow unimpeded access to the treatment site, said method comprising:
delivering a tissue traction device having a pair of legs biased with respect to each other by a biasing section to the treatment site;
engaging a tissue-engaging portion extending from one of the pair of legs of the tissue traction device with tissue to be drawn away from the treatment site;
engaging a tissue-engaging portion extending from the other of the pair of legs with tissue separate from the tissue to be drawn away from the treatment site; and
exerting a biasing force from the biasing section with respect to the legs of the tissue traction device to apply traction to the tissue to be drawn away from the treatment site.

33. The method of claim 32, wherein:
engaging a tissue-engaging portion of the other of the pair of legs comprises engaging the tissue-engaging portion of the other of the pair of legs with tissue at the treatment site and cut with respect to the tissue to be drawn away from the treatment site; and
exerting a biasing force comprises expanding the legs apart from each other via the biasing section.

34. The method of claim 32, wherein:
engaging a tissue-engaging portion of the other of the pair of legs comprises engaging the tissue-engaging portion of the other of the pair of legs with tissue spaced apart from the treatment site; and
exerting a biasing force comprises drawing the legs toward each other via the biasing section.

35. The method of claim 16, wherein engaging a tissue-engaging portion of at least one of the legs with tissue comprises engaging the tissue-engaging portion with tissue in a direction in which the biasing section exerts a biasing force with respect to the leg from which the tissue-engaging portion extends.
